(19) United States
(12) Patent Application Publication
(10) Pub. No.: US 2026/0283565 A1
(43) Pub. Date: Sep. 24, 2026

(54) SYSTEM FOR PROPOSING FIRST AID

(71) Applicant: SoftBank Group Corp., Tokyo (JP)

(72) Inventor: Taro KIMURA, Tokyo (JP)

(21) Appl. No.: 19/565,553

(22) Filed: Mar. 13, 2026

Related U.S. Application Data
(60) Provisional application No. 63/774,118, filed on Mar. 19, 2025.

(51) Int. Cl. A61B 5/00; A61B 5/16; G16H 80/00

(52) U.S. Cl. CPC A61B 5/747; A61B 5/0077; A61B 5/165; A61B 5/445; A61B 5/6803; A61B 5/7282; G16H 80/00

(57) ABSTRACT

A system that provides quick and appropriate first aid for non-urgent injuries and health problems. A user sends the content of an injury by text or a photograph using a messenger application via a terminal. A server analyzes the information using natural language processing and image recognition technology to determine a type and severity of the injury. Based on the analysis result, a first aid proposal unit proposes specific first aid such as a method of disinfection or use of a bandage. Furthermore, an urgency is evaluated using AI, and when a risk is high, a human operator is notified and provided with detailed instructions.

### CROSS-REFERENCE TO RELATED APPLICATIONS

This application is based upon and claims the benefit of priority from U.S. Provisional Patent Application No. 63/774118, filed on Mar. 19, 2025, the entire contents of which are incorporated herein by reference.

### BACKGROUND

Japanese Unexamined Patent Publication No. 2022-180282 discloses a method, which is a persona chatbot control method performed by at least one processor, the method including a step of receiving a user utterance, a step of adding the user utterance to a prompt including an instruction sentence associated with a description regarding a character of a chatbot, a step of encoding the prompt, and a step of inputting the encoded prompt to a language model to generate a chatbot utterance responding to the user utterance.

### SUMMARY

The problem to be solved by this disclosure is to reduce the burden on medical institutions and promote the efficient use of medical resources by providing appropriate first aid for non-urgent injuries and health problems. For example, unnecessary visits to medical institutions are reduced by enabling users to quickly receive appropriate first aid for minor injuries and health problems. This allows medical institutions to concentrate on more serious cases, and is expected to improve the quality of medical services as a whole. In addition, since users can receive first aid with peace of mind while at home, it becomes possible to receive appropriate medical support even in situations where access to medical institutions is difficult. In this way, disclosed herein is to alleviate the strain on medical institutions and to enable people to obtain appropriate medical treatment more quickly and safely.

Disclosed herein is a system including a message sending unit, an information analysis unit, a first aid proposal unit, an urgency evaluation unit, and an operator contact unit. The message sending unit provides an interface for receiving text messages and images related to injuries and health problems via a messenger application used by a user. The information analysis unit determines a type or severity of an injury by analyzing a received text message using natural language processing technology and analyzing an image using image recognition technology. The first aid proposal unit provides a means for proposing appropriate first aid to the user based on the type or severity of the injury determined by the information analysis unit. The urgency evaluation unit provides a means for inferring an urgency of the injury using AI and, when it is determined that a risk is high, contacting a human operator via the operator contact unit. The operator contact unit provides a means for notifying an operator having medical expertise of the user's situation and providing detailed instructions. This makes it possible for a user to quickly receive appropriate first aid, reduce the burden on medical institutions, and promote the efficient use of medical resources.

### BRIEF DESCRIPTION OF THE DRAWINGS

FIG. 1 is a conceptual diagram illustrating an example of a configuration of a data processing system according to a first embodiment.

FIG. 2 is a conceptual diagram illustrating an example of main functions of a data processing apparatus and a smart device according to the first embodiment.

FIG. 3 is a conceptual diagram illustrating an example of a configuration of a data processing system according to a second embodiment.

FIG. 4 is a conceptual diagram illustrating an example of main functions of a data processing apparatus and smart glasses according to the second embodiment.

FIG. 5 is a conceptual diagram illustrating an example of a configuration of a data processing system according to a third embodiment.

FIG. 6 is a conceptual diagram illustrating an example of main functions of a data processing apparatus and a headset-type terminal according to the third embodiment.

FIG. 7 is a conceptual diagram illustrating an example of a configuration of a data processing system according to a fourth embodiment.

FIG. 8 is a conceptual diagram illustrating an example of main functions of a data processing apparatus and a robot according to the fourth embodiment.

FIG. 9 illustrates an emotion map on which a plurality of emotions are mapped.

FIG. 10 illustrates an emotion map on which a plurality of emotions are mapped.

FIG. 11 is a flowchart illustrating an example method of proposing first aid.

### DETAILED DESCRIPTION

Hereinafter, example systems according to the technology of the present disclosure will be described with reference to the accompanying drawings.

First, terms used in the following description will be described.

In the following embodiments, a processor with a reference sign (hereinafter, simply referred to as a “processor”) may be one arithmetic device or may be a combination of a plurality of arithmetic devices. Also, the processor may be one type of arithmetic device or may be a combination of a plurality of types of arithmetic devices. Examples of the arithmetic device include a CPU (Central Processing Unit), a GPU (Graphics Processing Unit), a GPGPU (General-Purpose computing on Graphics Processing Units), an APU (Accelerated Processing Unit), and the like.

In the following embodiments, a RAM (Random Access Memory) with a reference sign is a memory in which information is temporarily stored, and is used as a work memory by a processor.

In the following embodiments, a storage with a reference sign is one or more non-volatile storage devices that store various programs, various parameters, and the like. Examples of the non-volatile storage device include a flash memory (SSD (Solid State Drive)), a magnetic disk (for example, a hard disk), or a magnetic tape, and the like.

In the following embodiments, a communication I/F (Interface) with a reference sign is an interface including a communication processor, an antenna, and the like. The communication I/F manages communication among a plurality of computers. An example of a communication standard applied to the communication I/F includes a wireless communication standard including 5G (5 th Generation Mobile Communication System), Wi-Fi (registered trademark), or Bluetooth (registered trademark), and the like.

In the following embodiments, “A and/or B” is synonymous with “at least one of A and B”. That is, “A and/or B” means that it may be A only, B only, or a combination of A and B. Also, in the present specification, when three or more matters are expressed by being connected with “and/or”, the same concept as “A and/or B” is applied.

### First Embodiment

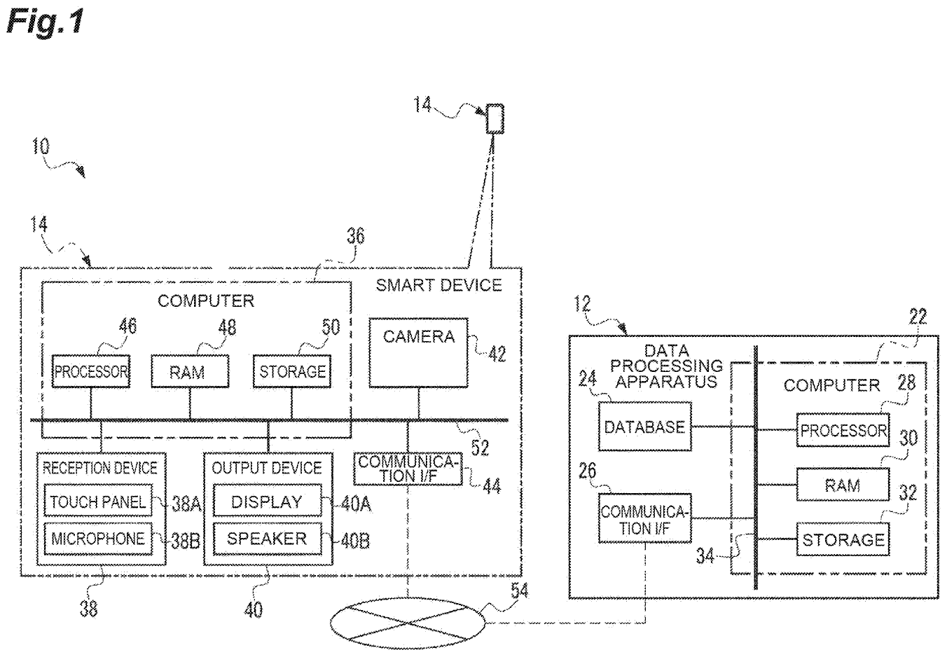

FIG. 1 illustrates an example of a configuration of a data processing system 10 according to a first embodiment.

As illustrated in FIG. 1, the data processing system 10 includes a data processing apparatus 12 and a smart device 14. An example of the data processing apparatus 12 includes a server.

The data processing apparatus 12 includes a computer 22, a database 24, and a communication I/F 26. The computer 22 is an example of a “computer” according to the technology of the present disclosure. The computer 22 includes a processor 28, a RAM 30, and a storage 32. The processor 28, the RAM 30, and the storage 32 are connected to a bus 34. Also, the database 24 and the communication I/F 26 are also connected to the bus 34. The communication I/F 26 is connected to a network 54. An example of the network 54 includes a WAN (Wide Area Network) and/or a LAN (Local Area Network), and the like.

The smart device 14 includes a computer 36, a reception device 38, an output device 40, a camera 42, and a communication I/F 44. The computer 36 includes a processor 46, a RAM 48, and a storage 50. The processor 46, the RAM 48, and the storage 50 are connected to a bus 52. Also, the reception device 38, the output device 40, and the camera 42 are also connected to the bus 52.

The reception device 38 includes a touch panel 38A, a microphone 38B, and the like, and receives a user input. The touch panel 38A receives a user input by contact of an indicator by detecting contact of the indicator (for example, a pen or a finger, etc.). The microphone 38B receives a user input by voice by detecting a user's voice. A control unit 46A transmits data indicating the user input received by the touch panel 38A and the microphone 38B to the data processing apparatus 12. In the data processing apparatus 12, a specific processing unit 290 acquires the data indicating the user input.

The output device 40 includes a display 40A, a speaker 40B, and the like, and presents data to a user 20 by outputting the data in a representation form (for example, voice and/or text) perceivable by the user 20. The display 40A displays visible information such as text and images in accordance with an instruction from the processor 46. The speaker 40B outputs voice in accordance with an instruction from the processor 46. The camera 42 is a small digital camera on which an optical system such as a lens, a diaphragm, and a shutter, and an image sensor such as a CMOS (Complementary Metal-Oxide-Semiconductor) image sensor or a CCD (Charge Coupled Device) image sensor are mounted.

The communication I/F 44 is connected to the network 54. The communication I/Fs 44 and 26 manage exchange of various information between the processor 46 and the processor 28 via the network 54.

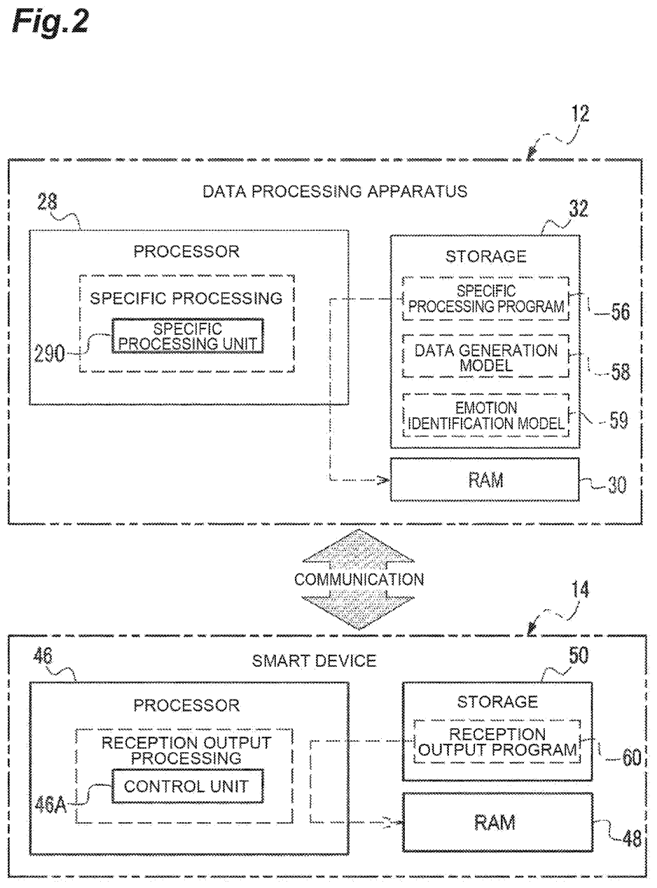

FIG. 2 illustrates an example of main functions of the data processing apparatus 12 and the smart device 14.

As illustrated in FIG. 2, in the data processing apparatus 12, specific processing is performed by the processor 28. A specific processing program 56 is stored in the storage 32. The specific processing program 56 is an example of a “program” according to the technology of the present disclosure. The processor 28 reads the specific processing program 56 from the storage 32 and executes the read specific processing program 56 on the RAM 30. The specific processing is realized by the processor 28 operating as a specific processing unit 290 in accordance with the specific processing program 56 executed on the RAM 30.

A data generation model 58 and an emotion identification model 59 are stored in the storage 32. The data generation model 58 and the emotion identification model 59 are used by the specific processing unit 290. The specific processing unit 290 can estimate a user's emotion using the emotion identification model 59 and perform specific processing using the user's emotion. In an emotion estimation function (emotion identification function) using the emotion identification model 59, various estimations and predictions regarding the user's emotion, including estimation and prediction of the user's emotion, are performed, but is not limited to such examples. Also, the estimation and prediction of emotion include, for example, analysis (analytics) of emotion and the like.

In the smart device 14, reception output processing is performed by the processor 46. A reception output program 60 is stored in the storage 50. The reception output program 60 is used in combination with the specific processing program 56 by the data processing system 10. The processor 46 reads the reception output program 60 from the storage 50 and executes the read reception output program 60 on the RAM 48. The specific processing is realized by the processor 46 operating as a control unit 46A in accordance with the reception output program 60 executed on the RAM 48. Note that the smart device 14 can also have a data generation model and an emotion identification model similar to the data generation model 58 and the emotion identification model 59, and perform processing similar to that of the specific processing unit 290 using these models. The reception output processing is realized by the processor 46 operating as the control unit 46A in accordance with the reception output program 60 executed on the RAM 48.

Note that an apparatus other than the data processing apparatus 12 may have the data generation model 58. For example, a server apparatus (for example, a generation server) may have the data generation model 58. In this case, the data processing apparatus 12 obtains a processing result (such as a prediction result) in which the data generation model 58 is used, by communicating with the server apparatus having the data generation model 58. Also, the data processing apparatus 12 may be a server apparatus, or may be a terminal device owned by a user (for example, a mobile phone, a robot, a home appliance, etc.). Next, an example of processing by the data processing system 10 according to the first embodiment will be described.

### Example 1.1

A flow of specific processing in Example 1.1 will be described. Each unit of the system described below is realized by the data processing apparatus 12 and the smart device 14. Also, the data processing apparatus 12 is referred to as a “server”, and the smart device 14 is referred to as a “terminal”.

Each unit of the system realized using a server and a terminal is realized by either the server or the terminal.

The system does not merely search for and present existing medical knowledge, but may organically couple hardware resources (camera sensor, microphone, GPS module) of a user terminal with computational resources on a server side.

First, a message sending unit is implemented on a user's terminal. The terminal is a device such as a smartphone, a tablet, or a personal computer, on which a messenger application that the user uses on a daily basis is installed. This application provides a means for inputting information regarding an injury or a health problem through a user interface. For example, if a user cuts a finger while cooking, the user can take a picture of the wound using the camera function of the terminal and enter “cut my finger” in a text box. In addition, the user can also use a voice input function to describe the symptoms by voice. This information is sent from the terminal to the server via the Internet.

The message sending unit may be configured by, for example, the computer 36 (processor 46, etc.), the reception device 38 (touch panel 38A, microphone 38B, etc.), and the communication I/F 44 of the smart device 14.

Next, an information analysis unit is implemented on the server. The server is a computer system provided with a high-performance processor and a large-capacity memory, and executes a plurality of algorithms for analyzing received data. Important keywords and context are extracted from a text message using natural language processing technology. For example, keywords such as “cut,” “bleeding,” and “pain” are identified, and the type of injury is specified based on these. In addition, image recognition technology is used to analyze the shape and color of the wound and the degree of bleeding from the sent photograph. For example, the length and depth of the wound and the amount of bleeding are quantified by image analysis, and the severity is evaluated based on these.

The information analysis unit may execute pixel-level segmentation processing on received image data using a deep learning model such as a Convolutional Neural Network (CNN), and vectorize a degree of oxidation based on an area and depth of a wound region and a hue of blood into numerical values. Unlike a human visual cognitive process, this processing extracts minute feature amounts indistinguishable by the naked eye by directly analyzing RGB values and point cloud data from a depth sensor (LiDAR, etc.).

The information analysis unit may perform noise removal and illumination correction as preprocessing on the received image data, and then apply a learned object detection model (for example, YOLOv8 or Faster R-CNN) to specify an affected part region (ROI: Region of Interest). Next, semantic segmentation may be performed on the specified ROI to classify pixels into classes of “skin,” “wound,” “blood,” and “foreign object.” Furthermore, an amount of bleeding may be estimated from the total number and distribution of blood pixels, and classification into an incised wound or a contused wound may be performed from shape features (aspect ratio, boundary complexity) of wound pixels. These processes are executed on the order of several milliseconds to several hundred milliseconds by the processor 28 (particularly a parallel arithmetic device such as a GPU or an NPU).

When performing natural language processing, the information analysis unit may tokenize a received text message and input it into a Transformer-based language model (for example, BERT or GPT architecture) to generate an embedding vector for each token. In this vector space, a cosine similarity with keywords indicating urgency such as “pain,” “bleeding,” and “clouding of consciousness” may be calculated to calculate an urgency score considering context. Thereby, a complex situation such as “it does not hurt but the bleeding does not stop,” which cannot be detected by simple keyword matching, is analyzed with high accuracy.

The information analysis unit may be configured by, for example, the computer 22 (processor 28, etc.) of the data processing apparatus 12, or may be configured by the specific processing unit 290, the specific processing program 56, the data generation model 58, and the like.

A first aid proposal unit is also implemented on the server. This unit receives data from the information analysis unit and generates optimal first aid based on a past medical database and expert knowledge. For example, for a minor cut, it generates instructions that explain in detail a method of cleaning with an antiseptic solution, a correct way to apply a bandage, a method of applying pressure to stop bleeding, and the like. Furthermore, the proposed content may be customized according to the user's age and medical history. These instructions are sent from the server to the terminal and displayed on the user's screen.

The first aid proposal unit may be configured by, for example, the computer 22 (processor 28, etc.) and the communication I/F 26 of the data processing apparatus 12, or may be configured by the specific processing unit 290, the specific processing program 56, the data generation model 58, and the like.

An urgency evaluation unit is also implemented on the server. This unit evaluates the urgency of an injury in real time using AI technology. For example, if bleeding is heavy or if it is determined that the wound is deep and the risk of infection is high, the urgency is evaluated as high. The AI refers to data from past similar cases and medical guidelines to make a quick and accurate judgment.

The urgency evaluation unit may collate the extracted feature amount vector with an anomaly detection model (for example, Isolation Forest or Autoencoder) learned from a vast past case database to calculate an anomaly score. Only when this score exceeds a predetermined threshold, the operator contact unit may establish a wideband and low-latency dedicated communication channel. This produces a technical effect of significantly reducing consumption of network bandwidth and shortening response time for truly highly urgent events compared to a case where an operator constantly monitors.

As an example of inference logic by the urgency evaluation unit, an evaluation method using multimodal learning may be adopted. For example, a “trauma severity vector” obtained from image analysis, a “subjective symptom vector” obtained from text analysis, and a “voice emotion vector (voice trembling, pitch, speech speed)” obtained from voice analysis (via the microphone 38B) can be concatenated to generate an integrated feature amount. This integrated feature amount can be input to a classifier for urgency determination (for example, LightGBM or Multilayer Perceptron) to output an urgency level (e.g., levels 1 to 5). At this time, in order to reduce false positives, reinforcement learning or adversarial learning using past misdiagnosis data may be incorporated.

The urgency evaluation unit may acquire user's vital data (heart rate, blood oxygen saturation acquired from a wearable device such as a smart watch) in real time and add it to the integrated feature amount. For example, logic (rule-based or machine learning-based) is incorporated that raises the urgency to the highest level assuming a possibility of internal bleeding or a shock state when a rapid increase in heart rate or a drop in blood pressure is detected even if the amount of bleeding on the image is small.

The urgency evaluation unit may be configured by, for example, the computer 22 (processor 28, etc.) of the data processing apparatus 12, or may be configured by the specific processing unit 290, the specific processing program 56, the data generation model 58, and the like.

An operator contact unit is implemented on the server. When the urgency evaluation unit determines a high urgency, the operator contact unit immediately sends a notification to a human operator. This operator has medical expertise and can provide direct support to the user. For example, the operator calls the user, confirms the detailed situation, and, if necessary, instructs the user to visit the nearest medical institution. In addition, the operator can also provide real-time guidance so that the user can correctly perform first aid.

In the operation of the operator contact unit, when it is determined that the urgency is high, the system may not only simply send a notification but also perform the following autonomous control. First, a P2P (Peer-to-Peer) video/voice communication session using WebRTC or the like is established between the user terminal and an operator terminal. Second, the analysis result (segmentation image of the affected part, estimated bleeding amount, history of vital data) generated by the information analysis unit is converted into a format conforming to a medical information exchange standard (for example, HL7 FHIR) and overlay-displayed on a screen of the operator terminal. Thereby, the operator can immediately grasp the situation immediately after connection, and the interview time can be shortened.

The system may have a function of automatically selecting a medical institution with the shortest transport time from the user's current location based on GPS information, and pre-transmitting patient information (including analysis results) via an API to a reservation system or an emergency reception system of the medical institution. At this time, for personal information protection, data is transmitted in an encrypted state using blockchain technology or homomorphic encryption.

The operator contact unit may be configured by, for example, the computer 22 (processor 28, etc.) and the communication I/F 26 of the data processing apparatus 12, or may be configured by the specific processing unit 290, the specific processing program 56, the data generation model 58, and the like.

In this way, A system that enables a user to quickly receive appropriate first aid through the cooperation of a terminal and a server is provided. This makes it possible to reduce the burden on medical institutions and promote the efficient use of medical resources. Furthermore, since users can receive first aid with peace of mind while at home, it becomes possible to receive appropriate medical support even in situations where access to medical institutions is difficult.

### System Configuration

The system includes a message sending unit, an information analysis unit, a first aid proposal unit, an urgency evaluation unit, and an operator contact unit. The message sending unit provides a means for a user to input information regarding an injury or a health problem via a terminal and send it to a server. For example, a user can input a text message or an image through a messenger application using a smartphone or a tablet. For example, if a user cuts a finger while cooking, the user can take a picture of the wound using the camera of the terminal and input text such as “cut my finger”. In addition, it is also possible to use a voice input function to describe the symptoms by voice. Furthermore, by having the user register past medical history and allergy information in advance, it becomes possible to propose first aid with higher accuracy.

The information analysis unit is implemented on a server and provides a means for analyzing received data. This unit extracts important keywords and context from a text message using advanced natural language processing technology. For example, it identifies keywords such as “cut,” “bleeding,” and “pain,” and specifies the type of injury based on these. In addition, it uses image recognition technology to analyze the shape and color of the wound and the degree of bleeding from the sent photograph. For example, it quantifies the length and depth of the wound and the amount of bleeding by image analysis, and evaluates the severity based on these. Furthermore, the analysis result is collated with a past medical database, and by referring to information on similar cases, a more accurate judgment becomes possible.

The first aid proposal unit receives data from the information analysis unit and provides a means for generating optimal first aid. This unit proposes specific first aid to a user based on past medical data and expert knowledge. For example, for a minor cut, it generates instructions that explain in detail a method of cleaning with an antiseptic solution, a correct way to apply a bandage, a method of applying pressure to stop bleeding, and the like. In addition, the proposed content may also be customized according to the user's age and medical history. Furthermore, the proposal presents a plurality of options, allowing the user to select the most suitable method.

The urgency evaluation unit provides a means for evaluating the urgency of an injury in real time using AI technology. This unit refers to data from past similar cases and medical guidelines to make a quick and accurate judgment. For example, if bleeding is heavy or if it is determined that the wound is deep and the risk of infection is high, the urgency is evaluated as high. Furthermore, the evaluation of urgency can be made more precise by also taking into account the user's current health condition and environmental conditions.

The operator contact unit provides a means for sending a notification to a human operator when the urgency evaluation unit determines a high urgency. This operator has medical expertise and can provide direct support to the user. For example, the operator calls the user, confirms the detailed situation, and, if necessary, instructs the user to visit the nearest medical institution. In addition, the operator can also provide real-time guidance so that the user can correctly perform first aid. Furthermore, it is also considered for the operator to provide psychological support to alleviate the user's anxiety.

Specific examples of prompt sentences to be read into the generative AI include “Please propose first aid for a case where a user has cut a finger.”, “Please evaluate the urgency for a case with heavy bleeding.”, and “Please provide guidance to an appropriate medical institution for the user.”. These prompt sentences are used as instructions for the AI to generate an appropriate response.

### IMPLEMENTATION STEPS

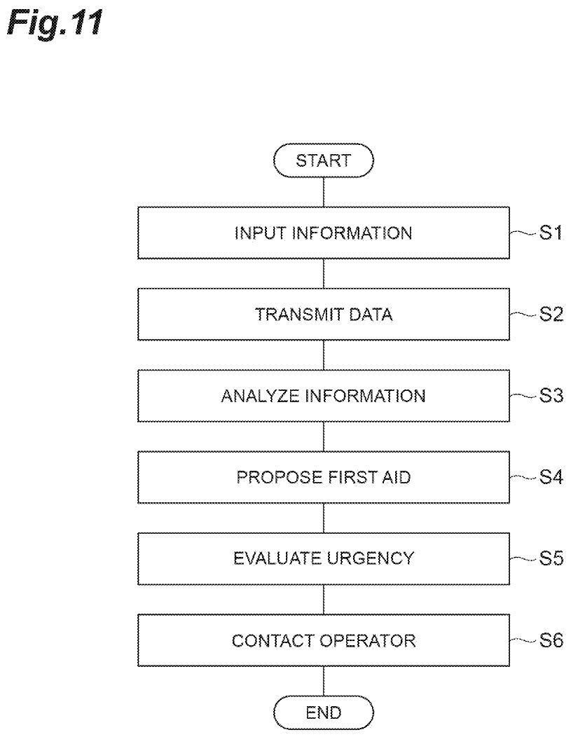

### Step 1: Information Input Step (Refer to Step S1 of FIG. 11)

A user inputs information regarding an injury or a health problem using a messenger application installed on a terminal. For example, the user takes a picture of a wound using a camera function of a smartphone or a tablet, and inputs a specific symptom such as “cut my finger” in a text box. In addition, it is also possible to use a voice input function to describe the symptoms by voice. This allows the user to provide information quickly and easily.

### Step 2: Data Sending Step (Refer to Step S2 of FIG. 11)

The input information is sent from the terminal to a server via the Internet. In this step, encryption technology is used to protect the information in order to maintain the accuracy and completeness of the data. This ensures the user's privacy and prevents data tampering.

### Step 3: Information Analysis Step (Refer to Step S3 of FIG. 11)

The data that has reached the server is analyzed by an information analysis unit. Important keywords and context are extracted from a text message using natural language processing technology. For example, keywords such as “cut,” “bleeding,” and “pain” are identified, and the type of injury is specified based on these. In addition, image recognition technology is used to analyze the shape and color of the wound and the degree of bleeding from the sent photograph. This quantifies the length and depth of the wound and the amount of bleeding, and evaluates the severity.

### Step 4: First Aid Proposal Step (Refer to Step S4 of FIG. 11)

Based on the data from the information analysis unit, a first aid proposal unit generates optimal first aid. Specific first aid is proposed to the user based on past medical data and expert knowledge. For example, for a minor cut, instructions are generated that explain in detail a method of cleaning with an antiseptic solution, a correct way to apply a bandage, a method of applying pressure to stop bleeding, and the like. A specific example of a prompt sentence to be read into a generative AI is “Please propose first aid for a case where a user has cut a finger.”.

### Step 5: Urgency Evaluation Step (Refer to Step S5 of FIG. 11)

The urgency of an injury is evaluated in real time using AI technology. A quick and accurate judgment is made by referring to data from past similar cases and medical guidelines. For example, if bleeding is heavy or if it is determined that the wound is deep and the risk of infection is high, the urgency is evaluated as high. A specific example of a prompt sentence to be read into a generative AI is “Please evaluate the urgency for a case with heavy bleeding.”.

### Step 6: Operator Contact Step (Refer to Step S6 of FIG. 11)

When the urgency evaluation unit determines a high urgency, an operator contact unit immediately sends a notification to a human operator. This operator has medical expertise and can provide direct support to the user. For example, the operator calls the user, confirms the detailed situation, and, if necessary, instructs the user to visit the nearest medical institution. In addition, the operator can also provide real-time guidance so that the user can correctly perform first aid. A specific example of a prompt sentence to be read into a generative AI is “Please provide guidance to an appropriate medical institution for the user.”.

### Specific Use Case

For example, consider a situation where a user accidentally cuts a finger while cooking at home. In this case, the user uses a smartphone to launch a messenger application installed on the terminal and takes a picture of the wound. Furthermore, the user inputs “Cut my finger. There is bleeding.” in a text box. This information is sent from the terminal to a server via the Internet.

The information that has reached the server is analyzed by an information analysis unit. Keywords such as “cut” and “bleeding” are extracted from the text message using natural language processing technology, and the shape of the wound and the degree of bleeding are analyzed from the photograph using image recognition technology. Based on the analysis result, a first aid proposal unit proposes appropriate first aid to the user. For example, it generates instructions that explain in detail a method of cleaning with an antiseptic solution, a correct way to apply a bandage, a method of applying pressure to stop bleeding, and the like.

Furthermore, an urgency evaluation unit evaluates the urgency of the injury using AI technology. If the amount of bleeding is large or if the wound is determined to be deep, the urgency is evaluated as high. In this case, an operator contact unit sends a notification to a human operator and provides direct support to the user. The operator calls the user, confirms the detailed situation, and, if necessary, instructs the user to visit the nearest medical institution.

Specific examples of prompt sentences to be read into the generative AI include “Please propose first aid for a case where a user has cut a finger.”, “Please evaluate the urgency for a case with heavy bleeding.”, and “Please provide guidance to an appropriate medical institution for the user.”. These prompt sentences are used as instructions for the AI to generate an appropriate response.

### Example 1.2

A flow of specific processing in Example 1.2 will be described. Each unit of the system described below is realized by the data processing apparatus 12 and the smart device 14. Also, the data processing apparatus 12 is referred to as a “server”, and the smart device 14 is referred to as a “terminal”.

A system for supporting health management and first aid for users in the nursing care field includes an information input unit, a data analysis unit, a first aid proposal unit, an urgency evaluation unit, and an expert contact unit.

First, the information input unit provides a means for care staff or users to input information on a user's health condition or injury using a terminal such as a smartphone or a tablet. This unit is designed to allow information to be input intuitively and quickly through a user interface. For example, if a user falls in daily life and suffers an abrasion on a knee, a staff member can take a picture of the wound using the camera function of the terminal. Furthermore, the staff member can input “abrasion on knee due to fall” in a text box and add detailed information such as the presence or absence of bleeding, the degree of pain, the time of occurrence, and the location. It is also possible to use a voice input function to describe the symptoms by voice, which allows information to be provided quickly even when hands are occupied or when it is difficult to input text.

Next, the data analysis unit is implemented on a server and analyzes the received information. This unit extracts keywords such as “fall,” “abrasion,” and “knee” from a text message and understands the context using advanced natural language processing technology. In addition, it analyzes the shape and color of the wound and the degree of bleeding from a photograph using image recognition technology. For example, it can quantify the length and depth of the wound and the amount of bleeding by image analysis and evaluate the severity. Furthermore, the analysis result is collated with a past medical database, and by referring to information on similar cases, a more accurate judgment becomes possible. This allows the user's condition to be grasped quickly and accurately, and an appropriate response to be made.

The first aid proposal unit provides a means for generating optimal first aid based on data from the data analysis unit. This unit proposes specific first aid to the user based on past medical data and expert knowledge. For example, for a minor abrasion, it generates instructions that explain in detail a method of cleaning with an antiseptic solution, a correct way to wrap with gauze or a bandage, a method of applying pressure to stop bleeding, and the like. In addition, the proposed content may also be customized according to the user's age, medical history, and allergy information. Furthermore, the proposal presents a plurality of options, allowing the care staff to select the most suitable method. This enables a flexible response according to the individual needs of the user.

The urgency evaluation unit provides a means for evaluating the urgency of an injury in real time using AI technology. This unit refers to data from past similar cases and medical guidelines to make a quick and accurate judgment. For example, if bleeding is heavy or if it is determined that the wound is deep and the risk of infection is high, the urgency is evaluated as high. Furthermore, the evaluation of urgency can be made more precise by also taking into account the user's current health condition and environmental conditions. This enables a quick and appropriate response even in an emergency, and ensures the user's safety.

The expert contact unit provides a means for sending a notification to a medical expert when the urgency evaluation unit determines a high urgency. This expert has medical expertise and can provide direct support to the user. For example, the expert calls the user, confirms the detailed situation, and, if necessary, instructs the user to visit the nearest medical institution. In addition, the expert can also provide real-time guidance so that the user can correctly perform first aid. Furthermore, it is also considered for the expert to provide psychological support to alleviate the user's anxiety. This allows the user to receive appropriate medical support with peace of mind.

In this way, A system that enables users to quickly receive appropriate first aid in nursing care facilities and home care through the cooperation of a terminal and a server is provided. This aims to reduce the burden on care staff and support users in leading a safer and more comfortable life.

### System Configuration

The system includes an information input unit, a data analysis unit, a first aid proposal unit, an urgency evaluation unit, and an expert contact unit. The information input unit provides a means for care staff or users to input information on a user's health condition or injury using a terminal such as a smartphone or a tablet. This unit is designed to allow information to be input intuitively and quickly through a user interface. For example, if a user falls in daily life and suffers an abrasion on a knee, a staff member can take a picture of the wound using the camera function of the terminal. Furthermore, the staff member can input “abrasion on knee due to fall” in a text box and add detailed information such as the presence or absence of bleeding, the degree of pain, the time of occurrence, and the location. It is also possible to use a voice input function to describe the symptoms by voice, which allows information to be provided quickly even when hands are occupied or when it is difficult to input text.

The data analysis unit is implemented on a server and analyzes the received information. This unit extracts keywords such as “fall,” “abrasion,” and “knee” from a text message and understands the context using advanced natural language processing technology. In addition, it analyzes the shape and color of the wound and the degree of bleeding from a photograph using image recognition technology. For example, it can quantify the length and depth of the wound and the amount of bleeding by image analysis and evaluate the severity. Furthermore, the analysis result is collated with a past medical database, and by referring to information on similar cases, a more accurate judgment becomes possible. This allows the user's condition to be grasped quickly and accurately, and an appropriate response to be made.

The first aid proposal unit provides a means for generating optimal first aid based on data from the data analysis unit. This unit proposes specific first aid to the user based on past medical data and expert knowledge. For example, for a minor abrasion, it generates instructions that explain in detail a method of cleaning with an antiseptic solution, a correct way to wrap with gauze or a bandage, a method of applying pressure to stop bleeding, and the like. In addition, the proposed content may also be customized according to the user's age, medical history, and allergy information. Furthermore, the proposal presents a plurality of options, allowing the care staff to select the most suitable method. This enables a flexible response according to the individual needs of the user.

The urgency evaluation unit provides a means for evaluating the urgency of an injury in real time using AI technology. This unit refers to data from past similar cases and medical guidelines to make a quick and accurate judgment. For example, if bleeding is heavy or if it is determined that the wound is deep and the risk of infection is high, the urgency is evaluated as high. Furthermore, the evaluation of urgency can be made more precise by also taking into account the user's current health condition and environmental conditions. This enables a quick and appropriate response even in an emergency, and ensures the user's safety.

The expert contact unit provides a means for sending a notification to a medical expert when the urgency evaluation unit determines a high urgency. This expert has medical expertise and can provide direct support to the user. For example, the expert calls the user, confirms the detailed situation, and, if necessary, instructs the user to visit the nearest medical institution. In addition, the expert can also provide real-time guidance so that the user can correctly perform first aid. Furthermore, it is also considered for the expert to provide psychological support to alleviate the user's anxiety. This allows the user to receive appropriate medical support with peace of mind.

Specific examples of prompt sentences to be read into the generative AI include “Please propose first aid for a case where a user has fallen.”, “Please evaluate the urgency of an abrasion.”, and “Please provide appropriate health management advice for the user.”. These prompt sentences are used as instructions for the AI to generate an appropriate response.

### IMPLEMENTATION STEPS

### Step 1: Information Input Step

In the information input step, a care staff or a user inputs information on the user's health condition or injury using a terminal such as a smartphone or a tablet. In this step, the user interface is designed to allow information to be input intuitively and quickly. For example, if a user falls in daily life and suffers an abrasion on a knee, a staff member can take a picture of the wound using the camera function of the terminal. Furthermore, the staff member can input “abrasion on knee due to fall” in a text box and add detailed information such as the presence or absence of bleeding, the degree of pain, the time of occurrence, and the location. It is also possible to use a voice input function to describe the symptoms by voice, which allows information to be provided quickly even when hands are occupied or when it is difficult to input text.

### Step 2: Data Sending Step

In the data sending step, the input information is sent from the terminal to a server via the Internet. In this step, encryption technology is used to protect the information in order to maintain the accuracy and completeness of the data. This ensures the user's privacy and prevents data tampering.

### Step 3: Information Analysis Step

In the information analysis step, the data that has reached the server is analyzed by a data analysis unit. Keywords such as “fall,” “abrasion,” and “knee” are extracted from a text message and the context is understood using natural language processing technology. In addition, the shape and color of the wound and the degree of bleeding are analyzed from a photograph using image recognition technology. For example, the length and depth of the wound and the amount of bleeding can be quantified by image analysis and the severity can be evaluated. Furthermore, the analysis result is collated with a past medical database, and by referring to information on similar cases, a more accurate judgment becomes possible.

### Step 4: First Aid Proposal Step

In the first aid proposal step, a first aid proposal unit generates optimal first aid based on data from the information analysis unit. Specific first aid is proposed to the user based on past medical data and expert knowledge. For example, for a minor abrasion, instructions are generated that explain in detail a method of cleaning with an antiseptic solution, a correct way to wrap with gauze or a bandage, a method of applying pressure to stop bleeding, and the like. A specific example of a prompt sentence to be read into a generative AI is “Please propose first aid for a case where a user has fallen.”.

### Step 5: Urgency Evaluation Step

In the urgency evaluation step, the urgency of an injury is evaluated in real time using AI technology. A quick and accurate judgment is made by referring to data from past similar cases and medical guidelines. For example, if bleeding is heavy or if it is determined that the wound is deep and the risk of infection is high, the urgency is evaluated as high. A specific example of a prompt sentence to be read into a generative AI is “Please evaluate the urgency of an abrasion.”.

### Step 6: Expert Contact Step

In the expert contact step, when the urgency evaluation unit determines a high urgency, an expert contact unit immediately sends a notification to a medical expert. This expert has medical expertise and can provide direct support to the user. For example, the expert calls the user, confirms the detailed situation, and, if necessary, instructs the user to visit the nearest medical institution. In addition, the expert can also provide real-time guidance so that the user can correctly perform first aid. A specific example of a prompt sentence to be read into a generative AI is “Please provide appropriate health management advice for the user.”.

### Specific Use Case

For example, consider a situation in a nursing care facility where a user falls during daily rehabilitation activities and suffers an abrasion on a knee. In this case, a care staff member quickly uses a smartphone to launch an application installed on the terminal and takes a picture of the wound. Furthermore, the staff member inputs “abrasion on knee due to fall during rehabilitation” in a text box and adds detailed information such as the presence or absence of bleeding, the degree of pain, the time of occurrence, and the location. This information is sent from the terminal to a server via the Internet.

The information that has reached the server is analyzed by a data analysis unit. Keywords such as “fall,” “abrasion,” and “knee” are extracted from the text message and the context is understood using natural language processing technology. In addition, the shape and color of the wound and the degree of bleeding are analyzed from the photograph using image recognition technology. Based on the analysis result, a first aid proposal unit proposes appropriate first aid to the care staff. For example, it generates instructions that explain in detail a method of cleaning with an antiseptic solution, a correct way to wrap with gauze or a bandage, a method of applying pressure to stop bleeding, and the like.

Furthermore, an urgency evaluation unit evaluates the urgency of the injury using AI technology. If bleeding is heavy or if it is determined that the wound is deep and the risk of infection is high, the urgency is evaluated as high. In this case, an expert contact unit immediately sends a notification to a medical expert and prompts a quick response. The medical expert can confirm the user's situation and, if necessary, instruct the user to visit a medical institution.

This system aims to reduce the burden on staff in nursing care facilities and to support users in leading a safer and more comfortable life. Specific examples of prompt sentences to be read into the generative AI include “Please propose first aid for a user who fell during rehabilitation.”, “Please evaluate the urgency of an abrasion.”, and “Please provide appropriate health management advice for the user.”. These prompt sentences are used as instructions for the AI to generate an appropriate response.

The specific processing unit 290 transmits a result of the specific processing to the smart device 14. In the smart device 14, the control unit 46A causes the output device 40 to output the result of the specific processing. The microphone 38B acquires voice indicating a user input for the result of the specific processing. The control unit 46A transmits voice data indicating the user input acquired by the microphone 38B to the data processing apparatus 12. In the data processing apparatus 12, the specific processing unit 290 acquires the voice data.

The data generation model 58 is a so-called generative AI (Artificial Intelligence). An example of the data generation model 58 includes a generative AI such as ChatGPT (registered trademark) (Internet search <URL: https://openai.com/blog/chatgpt>). The data generation model 58 is obtained by causing a neural network to perform deep learning. A prompt including an instruction is input to the data generation model 58, and inference data such as voice data indicating voice, text data indicating text, and image data indicating an image (for example, still image data or moving image data) is input. The data generation model 58 infers the input inference data in accordance with the instruction indicated by the prompt, and outputs an inference result in one or more data formats among voice data, text data, image data, and the like. The data generation model 58 includes, for example, a text generation AI, an image generation AI, a multimodal generation AI, and the like. Here, inference refers to, for example, analysis, classification, prediction, and/or summarization, and the like. The specific processing unit 290 performs the above-described specific processing while using the data generation model 58. The data generation model 58 may be a model fine-tuned to output an inference result from a prompt that does not include an instruction, and in this case, the data generation model 58 can output an inference result from a prompt that does not include an instruction. In the data processing apparatus 12 and the like, a plurality of types of data generation models 58 are included, and the data generation model 58 includes AIs other than generative AI. AIs other than generative AI are, for example, linear regression, logistic regression, a decision tree, a random forest, a support vector machine (SVM), k-means clustering, a convolutional neural network (CNN), a recurrent neural network (RNN), a generative adversarial network (GAN), or naive Bayes, and can perform various processes, but is not limited to such examples. Also, the AI may be an AI agent. Also, when the processing of each unit described above is performed by an AI, the processing is partially or entirely performed by the AI, but is not limited to such examples. Also, a process implemented by an AI including a generative AI may be replaced with a rule-based process, and a rule-based process may be replaced with a process implemented by an AI including a generative AI.

Also, the processing by the data processing system 10 described above is executed by the specific processing unit 290 of the data processing apparatus 12 or the control unit 46A of the smart device 14, but may also be executed by the specific processing unit 290 of the data processing apparatus 12 and the control unit 46A of the smart device 14. Also, the specific processing unit 290 of the data processing apparatus 12 acquires or collects information necessary for the processing from the smart device 14 or an external device, and the smart device 14 acquires or collects information necessary for the processing from the data processing apparatus 12 or an external device.

For example, a collection unit is realized by the control unit 46A of the smart device 14 or the specific processing unit 290 of the data processing apparatus 12. For example, an acquisition unit acquires step count data using the camera 42 or the communication I/F 44 of the smart device 14, and the data is processed by the specific processing unit 290 of the data processing apparatus 12. For example, an analysis unit is realized by the specific processing unit 290 of the data processing apparatus 12, and analyzes data from the collection unit and the acquisition unit. For example, a generation unit is realized by the specific processing unit 290 of the data processing apparatus 12, and generates a cooking menu using a generative AI. For example, a provision unit is realized by the output device 40 of the smart device 14 or the specific processing unit 290 of the data processing apparatus 12, and provides the generated cooking menu to a user. The correspondence relationship between each unit and the device or the control unit is not limited to the above-described example, and various changes are possible.

An example form in which the specific processing is performed by the data processing apparatus 12 has been described, but the technology of the present disclosure is not limited to this, and the specific processing may be performed by the smart device 14.

### Second Embodiment

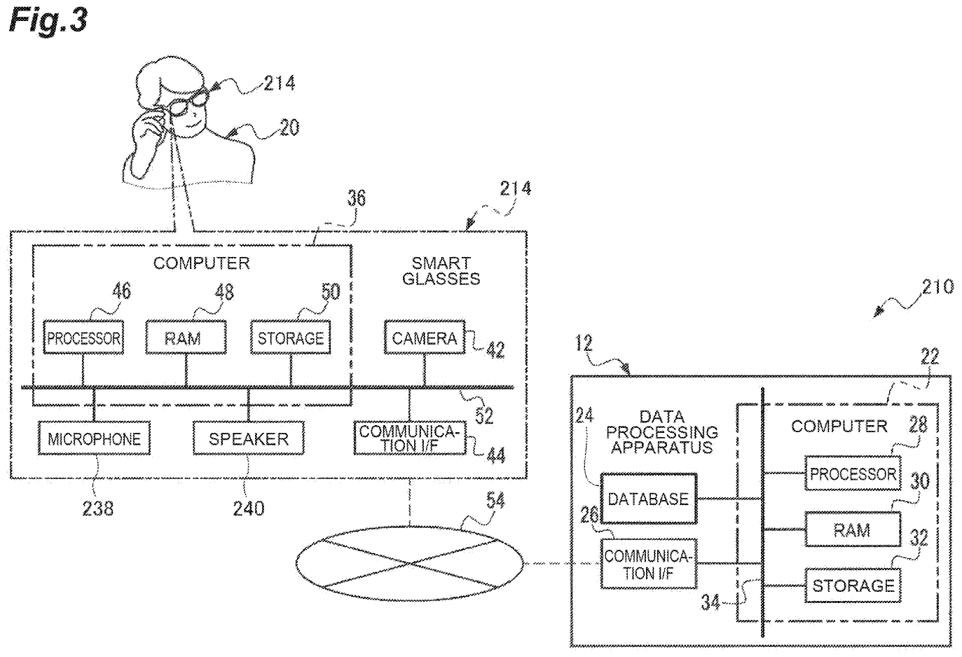

FIG. 3 illustrates an example of a configuration of a data processing system 210 according to a second embodiment.

As illustrated in FIG. 3, the data processing system 210 includes a data processing apparatus 12 and smart glasses 214. An example of the data processing apparatus 12 includes a server.

The data processing apparatus 12 includes a computer 22, a database 24, and a communication I/F 26. The computer 22 is an example of a “computer” according to the technology of the present disclosure. The computer 22 includes a processor 28, a RAM 30, and a storage 32. The processor 28, the RAM 30, and the storage 32 are connected to a bus 34. Also, the database 24 and the communication I/F 26 are also connected to the bus 34. The communication I/F 26 is connected to a network 54. An example of the network 54 includes a WAN (Wide Area Network) and/or a LAN (Local Area Network), and the like.

The smart glasses 214 include a computer 36, a microphone 238, a speaker 240, a camera 42, and a communication I/F 44. The computer 36 includes a processor 46, a RAM 48, and a storage 50. The processor 46, the RAM 48, and the storage 50 are connected to a bus 52. Also, the microphone 238, the speaker 240, and the camera 42 are also connected to the bus 52.

The microphone 238 receives an instruction or the like from a user 20 by receiving voice uttered by the user 20. The microphone 238 captures the voice uttered by the user 20, converts the captured voice into voice data, and outputs the voice data to the processor 46. The speaker 240 outputs voice in accordance with an instruction from the processor 46.

The camera 42 is a small digital camera on which an optical system such as a lens, a diaphragm, and a shutter, and an image sensor such as a CMOS (Complementary Metal-Oxide-Semiconductor) image sensor or a CCD (Charge Coupled Device) image sensor are mounted, and images the surroundings of the user 20 (for example, an imaging range defined by an angle of view corresponding to the width of the field of view of a general person with normal vision).

The communication I/F 44 is connected to the network 54. The communication I/Fs 44 and 26 manage exchange of various information between the processor 46 and the processor 28 via the network 54. The exchange of various information between the processor 46 and the processor 28 using the communication I/Fs 44 and 26 is performed in a secure state.

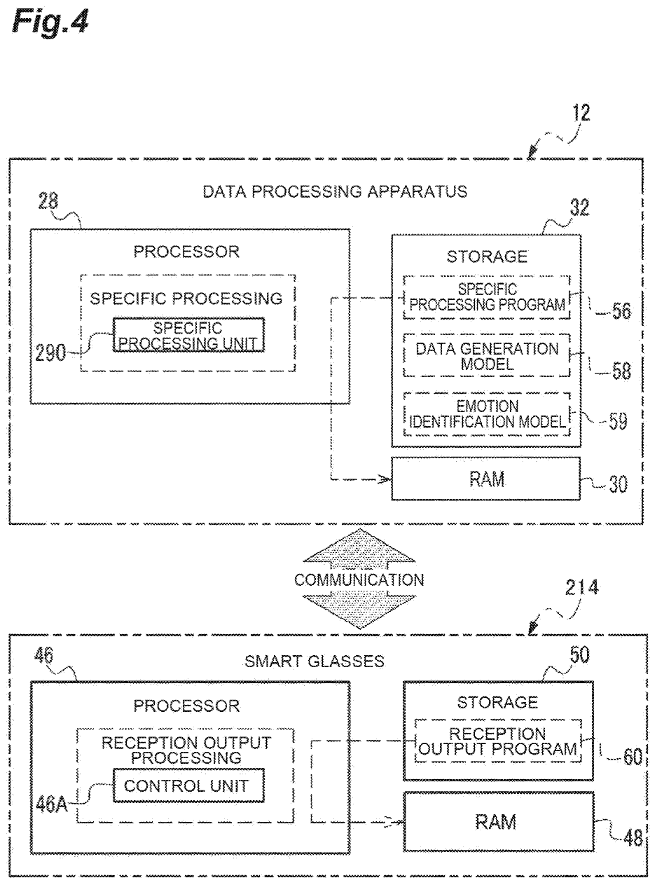

FIG. 4 illustrates an example of main functions of the data processing apparatus 12 and the smart glasses 214. As illustrated in FIG. 4, in the data processing apparatus 12, specific processing is performed by the processor 28. A specific processing program 56 is stored in the storage 32.

The specific processing program 56 is an example of a “program” according to the technology of the present disclosure. The processor 28 reads the specific processing program 56 from the storage 32 and executes the read specific processing program 56 on the RAM 30. The specific processing is realized by the processor 28 operating as a specific processing unit 290 in accordance with the specific processing program 56 executed on the RAM 30.

A data generation model 58 and an emotion identification model 59 are stored in the storage 32. The data generation model 58 and the emotion identification model 59 are used by the specific processing unit 290. The specific processing unit 290 can estimate a user's emotion using the emotion identification model 59 and perform specific processing using the user's emotion. In an emotion estimation function (emotion identification function) using the emotion identification model 59, various estimations and predictions regarding the user's emotion, including estimation and prediction of the user's emotion, are performed, but is not limited to such examples. Also, the estimation and prediction of emotion include, for example, analysis (analytics) of emotion and the like.

In the smart glasses 214, reception output processing is performed by the processor 46. A reception output program 60 is stored in the storage 50. The processor 46 reads the reception output program 60 from the storage 50 and executes the read reception output program 60 on the RAM 48. The reception output processing is realized by the processor 46 operating as a control unit 46A in accordance with the reception output program 60 executed on the RAM 48. Note that the smart glasses 214 can also have a data generation model and an emotion identification model similar to the data generation model 58 and the emotion identification model 59, and perform processing similar to that of the specific processing unit 290 using these models.

Next, specific processing by the specific processing unit 290 of the data processing apparatus 12 will be described. Each unit of the system described below is realized by the data processing apparatus 12 and the smart glasses 214. In the following description, the data processing apparatus 12 is referred to as a “server”, and the smart glasses 214 are referred to as a “terminal”.

### Example 2.1

Since the flow of the specific processing is the same as that in Example 1.1 described in the first embodiment, a description thereof is omitted.

### Example 2.2

Since the flow of the specific processing is the same as that in Example 1.2 described in the first embodiment, a description thereof is omitted.

The specific processing unit 290 transmits a result of the specific processing to the smart glasses 214. In the smart glasses 214, the control unit 46A causes the speaker 240 to output the result of the specific processing. The microphone 238 acquires voice indicating a user input for the result of the specific processing. The control unit 46A transmits voice data indicating the user input acquired by the microphone 238 to the data processing apparatus 12. In the data processing apparatus 12, the specific processing unit 290 acquires the voice data.

The data generation model 58 is a so-called generative AI (Artificial Intelligence). An example of the data generation model 58 includes a generative AI such as ChatGPT (registered trademark) (Internet search <URL: https://openai.com/blog/chatgpt>). The data generation model 58 is obtained by causing a neural network to perform deep learning. A prompt including an instruction is input to the data generation model 58, and inference data such as voice data indicating voice, text data indicating text, and image data indicating an image (for example, still image data or moving image data) is input. The data generation model 58 infers the input inference data in accordance with the instruction indicated by the prompt, and outputs an inference result in one or more data formats among voice data, text data, image data, and the like. The data generation model 58 includes, for example, a text generation AI, an image generation AI, a multimodal generation AI, and the like. Here, inference refers to, for example, analysis, classification, prediction, and/or summarization, and the like. The specific processing unit 290 performs the above-described specific processing while using the data generation model 58. The data generation model 58 may be a model fine-tuned to output an inference result from a prompt that does not include an instruction, and in this case, the data generation model 58 can output an inference result from a prompt that does not include an instruction. In the data processing apparatus 12 and the like, a plurality of types of data generation models 58 are included, and the data generation model 58 includes AIs other than generative AI. AIs other than generative AI are, for example, linear regression, logistic regression, a decision tree, a random forest, a support vector machine (SVM), k-means clustering, a convolutional neural network (CNN), a recurrent neural network (RNN), a generative adversarial network (GAN), or naive Bayes, and can perform various processes, but is not limited to such examples. Also, the AI may be an AI agent. Also, when the processing of each unit described above is performed by an AI, the processing is partially or entirely performed by the AI, but is not limited to such examples. Also, a process implemented by an AI including a generative AI may be replaced with a rule-based process, and a rule-based process may be replaced with a process implemented by an AI including a generative AI.

Also, the processing by the data processing system 10 described above is executed by the specific processing unit 290 of the data processing apparatus 12 or the control unit 46A of the smart device 14, but may also be executed by the specific processing unit 290 of the data processing apparatus 12 and the control unit 46A of the smart device 14. Also, the specific processing unit 290 of the data processing apparatus 12 acquires or collects information necessary for the processing from the smart device 14 or an external device, and the smart device 14 acquires or collects information necessary for the processing from the data processing apparatus 12 or an external device.

For example, a collection unit is realized by the control unit 46A of the smart device 14 or the specific processing unit 290 of the data processing apparatus 12. For example, an acquisition unit acquires step count data using the camera 42 or the communication I/F 44 of the smart device 14, and the data is processed by the specific processing unit 290 of the data processing apparatus 12. For example, an analysis unit is realized by the specific processing unit 290 of the data processing apparatus 12, and analyzes data from the collection unit and the acquisition unit. For example, a generation unit is realized by the specific processing unit 290 of the data processing apparatus 12, and generates a cooking menu using a generative AI. For example, a provision unit is realized by the output device 40 of the smart device 14 or the specific processing unit 290 of the data processing apparatus 12, and provides the generated cooking menu to a user. The correspondence relationship between each unit and the device or the control unit is not limited to the above-described example, and various changes are possible.

An example form in which the specific processing is performed by the data processing apparatus 12 has been described, but the technology of the present disclosure is not limited to this, and the specific processing may be performed by the smart glasses 214.

### Third Embodiment

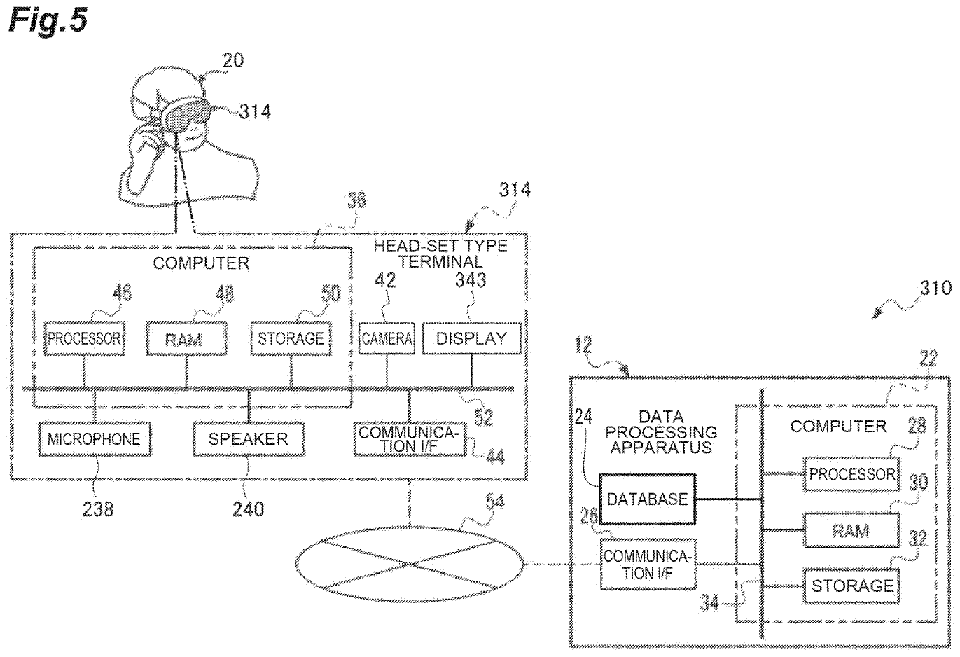

FIG. 5 illustrates an example of a configuration of a data processing system 310 according to a third embodiment.

As illustrated in FIG. 5, the data processing system 310 includes a data processing apparatus 12 and a headset-type terminal 314. An example of the data processing apparatus 12 includes a server.

The data processing apparatus 12 includes a computer 22, a database 24, and a communication I/F 26. The computer 22 is an example of a “computer” according to the technology of the present disclosure. The computer 22 includes a processor 28, a RAM 30, and a storage 32. The processor 28, the RAM 30, and the storage 32 are connected to a bus 34. Also, the database 24 and the communication I/F 26 are also connected to the bus 34. The communication I/F 26 is connected to a network 54. An example of the network 54 includes a WAN (Wide Area Network) and/or a LAN (Local Area Network), and the like.

The headset-type terminal 314 includes a computer 36, a microphone 238, a speaker 240, a camera 42, a communication I/F 44, and a display 343. The computer 36 includes a processor 46, a RAM 48, and a storage 50. The processor 46, the RAM 48, and the storage 50 are connected to a bus 52. Also, the microphone 238, the speaker 240, the camera 42, and the display 343 are also connected to the bus 52.

The microphone 238 receives an instruction or the like from a user 20 by receiving voice uttered by the user 20. The microphone 238 captures the voice uttered by the user 20, converts the captured voice into voice data, and outputs the voice data to the processor 46. The speaker 240 outputs voice in accordance with an instruction from the processor 46.

The camera 42 is a small digital camera on which an optical system such as a lens, a diaphragm, and a shutter, and an image sensor such as a CMOS (Complementary Metal-Oxide-Semiconductor) image sensor or a CCD (Charge Coupled Device) image sensor are mounted, and images the surroundings of the user 20 (for example, an imaging range defined by an angle of view corresponding to the width of the field of view of a general person with normal vision).

The communication I/F 44 is connected to the network 54. The communication I/Fs 44 and 26 manage exchange of various information between the processor 46 and the processor 28 via the network 54. The exchange of various information between the processor 46 and the processor 28 using the communication I/Fs 44 and 26 is performed in a secure state.

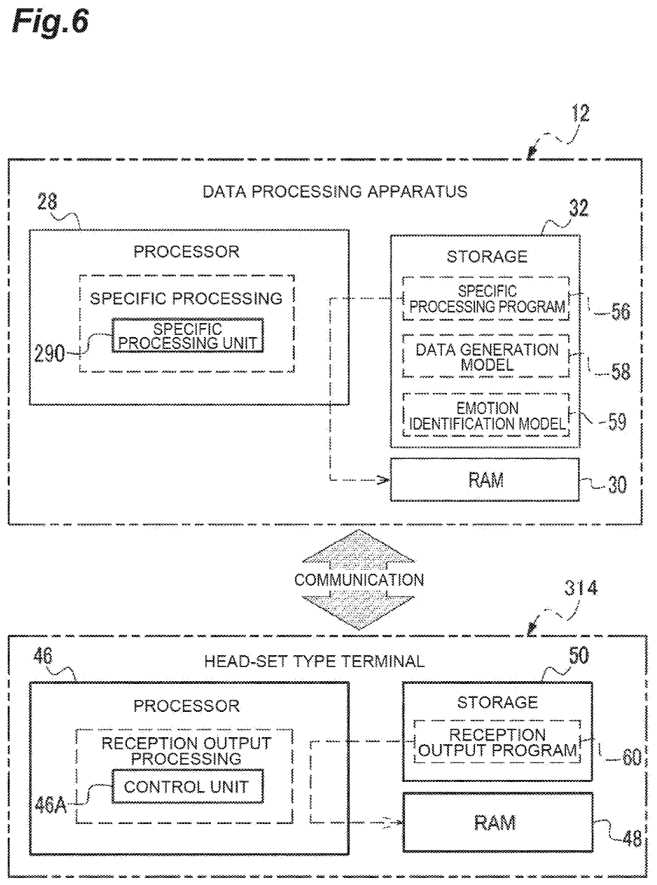

FIG. 6 illustrates an example of main functions of the data processing apparatus 12 and the headset-type terminal 314. As illustrated in FIG. 6, in the data processing apparatus 12, specific processing is performed by the processor 28. A specific processing program 56 is stored in the storage 32.

The specific processing program 56 is an example of a “program” according to the technology of the present disclosure. The processor 28 reads the specific processing program 56 from the storage 32 and executes the read specific processing program 56 on the RAM 30. The specific processing is realized by the processor 28 operating as a specific processing unit 290 in accordance with the specific processing program 56 executed on the RAM 30.

A data generation model 58 and an emotion identification model 59 are stored in the storage 32. The data generation model 58 and the emotion identification model 59 are used by the specific processing unit 290.

In the headset-type terminal 314, reception output processing is performed by the processor 46. A reception output program 60 is stored in the storage 50. The processor 46 reads the reception output program 60 from the storage 50 and executes the read reception output program 60 on the RAM 48. The reception output processing is realized by the processor 46 operating as a control unit 46A in accordance with the reception output program 60 executed on the RAM 48.

Next, specific processing by the specific processing unit 290 of the data processing apparatus 12 will be described. Each unit of the system described below is realized by the data processing apparatus 12 and the headset-type terminal 314. In the following description, the data processing apparatus 12 is referred to as a “server”, and the headset-type terminal 314 is referred to as a “terminal”.

### Example 3.1

Since the flow of the specific processing is the same as that in Example 1.1 described in the first embodiment, a description thereof is omitted.

### Example 3.2

Since the flow of the specific processing is the same as that in Example 1.2 described in the first embodiment, a description thereof is omitted.

The specific processing unit 290 transmits a result of the specific processing to the headset-type terminal 314. In the headset-type terminal 314, the control unit 46A causes the speaker 240 and the display 343 to output the result of the specific processing. The microphone 238 acquires voice indicating a user input for the result of the specific processing. The control unit 46A transmits voice data indicating the user input acquired by the microphone 238 to the data processing apparatus 12. In the data processing apparatus 12, the specific processing unit 290 acquires the voice data.

The data generation model 58 is a so-called generative AI (Artificial Intelligence). An example of the data generation model 58 includes a generative AI such as ChatGPT (registered trademark) (Internet search <URL: https://openai.com/blog/chatgpt>). The data generation model 58 is obtained by causing a neural network to perform deep learning. A prompt including an instruction is input to the data generation model 58, and inference data such as voice data indicating voice, text data indicating text, and image data indicating an image (for example, still image data or moving image data) is input. The data generation model 58 infers the input inference data in accordance with the instruction indicated by the prompt, and outputs an inference result in one or more data formats among voice data, text data, image data, and the like. The data generation model 58 includes, for example, a text generation AI, an image generation AI, a multimodal generation AI, and the like. Here, inference refers to, for example, analysis, classification, prediction, and/or summarization, and the like. The specific processing unit 290 performs the above-described specific processing while using the data generation model 58. The data generation model 58 may be a model fine-tuned to output an inference result from a prompt that does not include an instruction, and in this case, the data generation model 58 can output an inference result from a prompt that does not include an instruction. In the data processing apparatus 12 and the like, a plurality of types of data generation models 58 are included, and the data generation model 58 includes AIs other than generative AI. AIs other than generative AI are, for example, linear regression, logistic regression, a decision tree, a random forest, a support vector machine (SVM), k-means clustering, a convolutional neural network (CNN), a recurrent neural network (RNN), a generative adversarial network (GAN), or naive Bayes, and can perform various processes, but is not limited to such examples. Also, the AI may be an AI agent. Also, when the processing of each unit described above is performed by an AI, the processing is partially or entirely performed by the AI, but is not limited to such examples. Also, a process implemented by an AI including a generative AI may be replaced with a rule-based process, and a rule-based process may be replaced with a process implemented by an AI including a generative AI.

Also, the processing by the data processing system 10 described above is executed by the specific processing unit 290 of the data processing apparatus 12 or the control unit 46A of the smart device 14, but may also be executed by the specific processing unit 290 of the data processing apparatus 12 and the control unit 46A of the smart device 14. Also, the specific processing unit 290 of the data processing apparatus 12 acquires or collects information necessary for the processing from the smart device 14 or an external device, and the smart device 14 acquires or collects information necessary for the processing from the data processing apparatus 12 or an external device.

For example, a collection unit is realized by the control unit 46A of the smart device 14 or the specific processing unit 290 of the data processing apparatus 12. For example, an acquisition unit acquires step count data using the camera 42 or the communication I/F 44 of the smart device 14, and the data is processed by the specific processing unit 290 of the data processing apparatus 12. For example, an analysis unit is realized by the specific processing unit 290 of the data processing apparatus 12, and analyzes data from the collection unit and the acquisition unit. For example, a generation unit is realized by the specific processing unit 290 of the data processing apparatus 12, and generates a cooking menu using a generative AI. For example, a provision unit is realized by the output device 40 of the smart device 14 or the specific processing unit 290 of the data processing apparatus 12, and provides the generated cooking menu to a user. The correspondence relationship between each unit and the device or the control unit is not limited to the above-described example, and various changes are possible.

An example form in which the specific processing is performed by the data processing apparatus 12 has been described, but the technology of the present disclosure is not limited to this, and the specific processing may be performed by the headset-type terminal 314.

### Fourth Embodiment

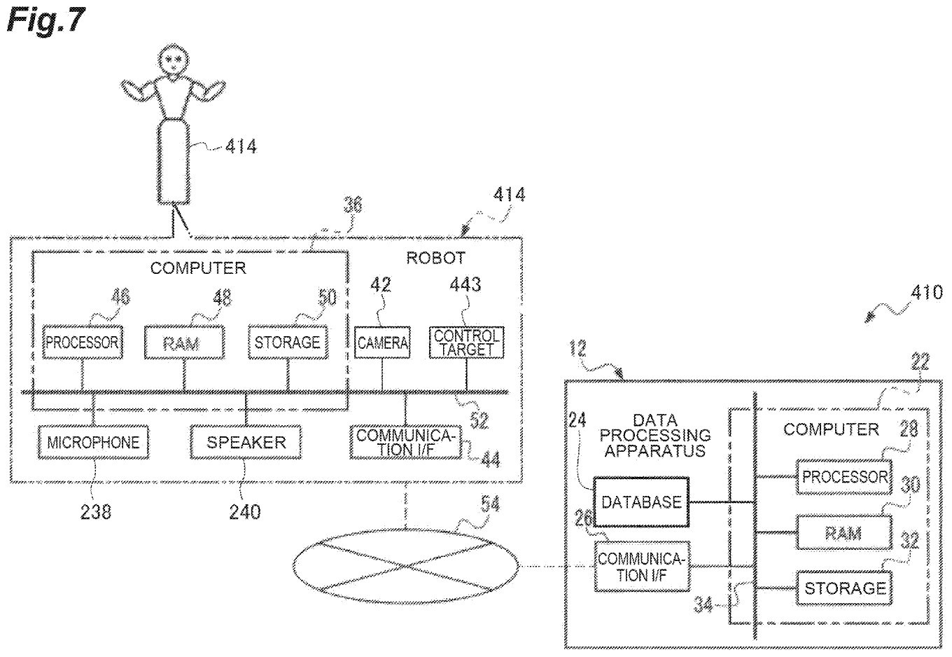

FIG. 7 illustrates an example of a configuration of a data processing system 410 according to a fourth embodiment.

As illustrated in FIG. 7, the data processing system 410 includes a data processing apparatus 12 and a robot 414. An example of the data processing apparatus 12 includes a server.

The data processing apparatus 12 includes a computer 22, a database 24, and a communication I/F 26. The computer 22 is an example of a “computer” according to the technology of the present disclosure. The computer 22 includes a processor 28, a RAM 30, and a storage 32. The processor 28, the RAM 30, and the storage 32 are connected to a bus 34. Also, the database 24 and the communication I/F 26 are also connected to the bus 34. The communication I/F 26 is connected to a network 54. An example of the network 54 includes a WAN (Wide Area Network) and/or a LAN (Local Area Network), and the like.

The robot 414 includes a computer 36, a microphone 238, a speaker 240, a camera 42, a communication I/F 44, and a control target 443. The computer 36 includes a processor 46, a RAM 48, and a storage 50. The processor 46, the RAM 48, and the storage 50 are connected to a bus 52. Also, the microphone 238, the speaker 240, the camera 42, and the control target 443 are also connected to the bus 52.

The microphone 238 receives an instruction or the like from a user 20 by receiving voice uttered by the user 20. The microphone 238 captures the voice uttered by the user 20, converts the captured voice into voice data, and outputs the voice data to the processor 46. The speaker 240 outputs voice in accordance with an instruction from the processor 46.

The camera 42 is a small digital camera on which an optical system such as a lens, a diaphragm, and a shutter, and an image sensor such as a CMOS (Complementary Metal-Oxide-Semiconductor) image sensor or a CCD (Charge Coupled Device) image sensor are mounted, and images the surroundings of the user 20 (for example, an imaging range defined by an angle of view corresponding to the width of the field of view of a general person with normal vision).

The communication I/F 44 is connected to the network 54. The communication I/Fs 44 and 26 manage exchange of various information between the processor 46 and the processor 28 via the network 54. The exchange of various information between the processor 46 and the processor 28 using the communication I/Fs 44 and 26 is performed in a secure state.

The control target 443 includes a display device, an LED of an eye part, and motors that drive an arm, a hand, a leg, and the like. The posture and gestures of the robot 414 are controlled by controlling the motors of the arm, hand, leg, and the like. A part of the emotions of the robot 414 can be expressed by controlling these motors. Also, the facial expression of the robot 414 can also be expressed by controlling the light emission state of the LED of the eye part of the robot 414.

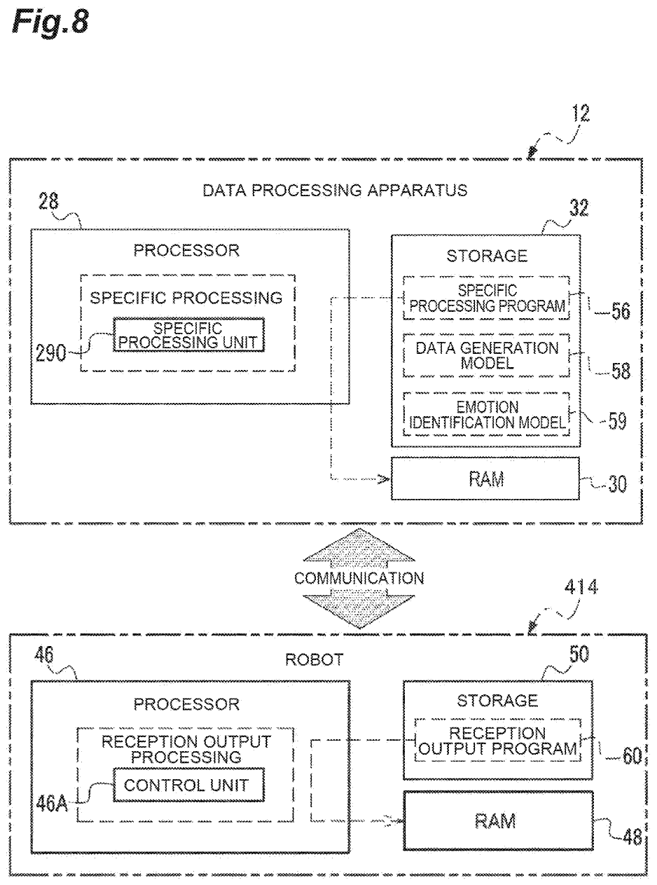

FIG. 8 illustrates an example of main functions of the data processing apparatus 12 and the robot 414. As illustrated in FIG. 8, in the data processing apparatus 12, specific processing is performed by the processor 28. A specific processing program 56 is stored in the storage 32.

The specific processing program 56 is an example of a “program” according to the technology of the present disclosure. The processor 28 reads the specific processing program 56 from the storage 32 and executes the read specific processing program 56 on the RAM 30. The specific processing is realized by the processor 28 operating as a specific processing unit 290 in accordance with the specific processing program 56 executed on the RAM 30.

A data generation model 58 and an emotion identification model 59 are stored in the storage 32. The data generation model 58 and the emotion identification model 59 are used by the specific processing unit 290.

In the robot 414, reception output processing is performed by the processor 46. A reception output program 60 is stored in the storage 50. The processor 46 reads the reception output program 60 from the storage 50 and executes the read reception output program 60 on the RAM 48. The reception output processing is realized by the processor 46 operating as a control unit 46A in accordance with the reception output program 60 executed on the RAM 48.

Next, specific processing by the specific processing unit 290 of the data processing apparatus 12 will be described. Each unit of the system described below is realized by the data processing apparatus 12 and the robot 414. In the following description, the data processing apparatus 12 is referred to as a “server”, and the robot 414 is referred to as a “terminal”.

### Example 4.1

Since the flow of the specific processing is the same as that in Example 1.1 described in the first embodiment, a description thereof is omitted.

### Example 4.2

Since the flow of the specific processing is the same as that in Example 1.2 described in the first embodiment, a description thereof is omitted.

The specific processing unit 290 transmits a result of the specific processing to the robot 414. In the robot 414, the control unit 46A causes the speaker 240 and the control target 443 to output the result of the specific processing. The microphone 238 acquires voice indicating a user input for the result of the specific processing. The control unit 46A transmits voice data indicating the user input acquired by the microphone 238 to the data processing apparatus 12. In the data processing apparatus 12, the specific processing unit 290 acquires the voice data.

The data generation model 58 is a so-called generative AI (Artificial Intelligence). An example of the data generation model 58 includes a generative AI such as ChatGPT (registered trademark) (Internet search <URL: https://openai.com/blog/chatgpt>). The data generation model 58 is obtained by causing a neural network to perform deep learning. A prompt including an instruction is input to the data generation model 58, and inference data such as voice data indicating voice, text data indicating text, and image data indicating an image (for example, still image data or moving image data) is input. The data generation model 58 infers the input inference data in accordance with the instruction indicated by the prompt, and outputs an inference result in one or more data formats among voice data, text data, image data, and the like. The data generation model 58 includes, for example, a text generation AI, an image generation AI, a multimodal generation AI, and the like. Here, inference refers to, for example, analysis, classification, prediction, and/or summarization, and the like. The specific processing unit 290 performs the above-described specific processing while using the data generation model 58. The data generation model 58 may be a model fine-tuned to output an inference result from a prompt that does not include an instruction, and in this case, the data generation model 58 can output an inference result from a prompt that does not include an instruction. In the data processing apparatus 12 and the like, a plurality of types of data generation models 58 are included, and the data generation model 58 includes AIs other than generative AI. AIs other than generative AI are, for example, linear regression, logistic regression, a decision tree, a random forest, a support vector machine (SVM), k-means clustering, a convolutional neural network (CNN), a recurrent neural network (RNN), a generative adversarial network (GAN), or naive Bayes, and can perform various processes, but is not limited to such examples. Also, the AI may be an AI agent. Also, when the processing of each unit described above is performed by an AI, the processing is partially or entirely performed by the AI, but is not limited to such examples. Also, a process implemented by an AI including a generative AI may be replaced with a rule-based process, and a rule-based process may be replaced with a process implemented by an AI including a generative AI.

Also, the processing by the data processing system 10 described above is executed by the specific processing unit 290 of the data processing apparatus 12 or the control unit 46A of the smart device 14, but may also be executed by the specific processing unit 290 of the data processing apparatus 12 and the control unit 46A of the smart device 14. Also, the specific processing unit 290 of the data processing apparatus 12 acquires or collects information necessary for the processing from the smart device 14 or an external device, and the smart device 14 acquires or collects information necessary for the processing from the data processing apparatus 12 or an external device.

For example, a collection unit is realized by the control unit 46A of the smart device 14 or the specific processing unit 290 of the data processing apparatus 12. For example, an acquisition unit acquires step count data using the camera 42 or the communication I/F 44 of the smart device 14, and the data is processed by the specific processing unit 290 of the data processing apparatus 12. For example, an analysis unit is realized by the specific processing unit 290 of the data processing apparatus 12, and analyzes data from the collection unit and the acquisition unit. For example, a generation unit is realized by the specific processing unit 290 of the data processing apparatus 12, and generates a cooking menu using a generative AI. For example, a provision unit is realized by the output device 40 of the smart device 14 or the specific processing unit 290 of the data processing apparatus 12, and provides the generated cooking menu to a user. The correspondence relationship between each unit and the device or the control unit is not limited to the above-described example, and various changes are possible.

An example form in which the specific processing is performed by the data processing apparatus 12 has been described, but the technology of the present disclosure is not limited to this, and the specific processing may be performed by the robot 414.

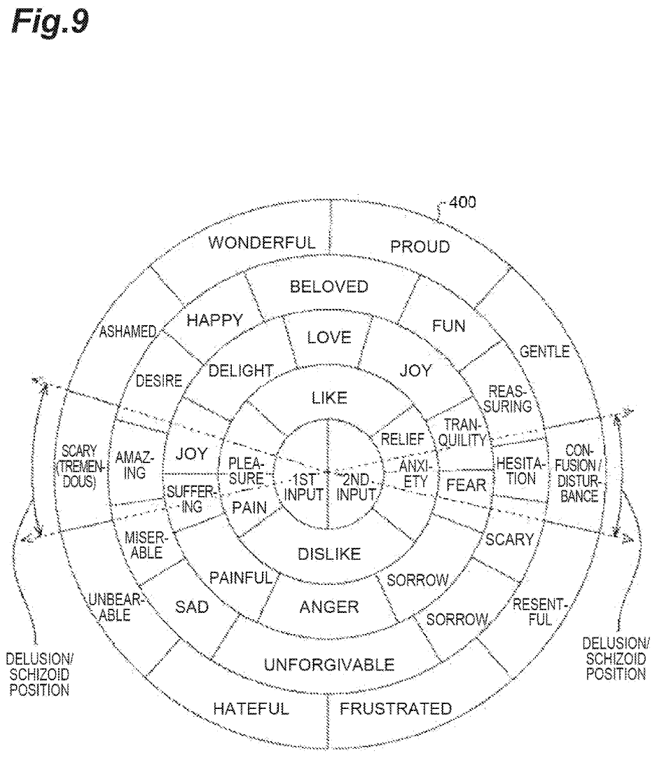

Note that the emotion identification model 59 as an emotion engine may determine a user's emotion according to a specific mapping. For example, the emotion identification model 59 may determine the user's emotion according to an emotion map (see FIG. 9), which is a specific mapping. Also, the emotion identification model 59 may similarly determine the robot's emotion, and the specific processing unit 290 may perform specific processing using the robot's emotion.

In the robot 414, when the urgency evaluation unit determines that “the user cannot perform first aid by themselves” and “the urgency is high,” the control unit 46A may drive the control target 443 (manipulator, etc.) to shift to a mode for performing physical first aid support. For example, an operation of specifying a bleeding site by image recognition and compressing and stopping bleeding of the affected part using a robot arm, or an operation of transporting an AED (Automated External Defibrillator) and guiding attachment can be executed. At this time, a motion plan (Motion Planning) of the robot can be generated based on inverse kinematics calculation while tracking the position of the affected part and the posture of the user in real time.

FIG. 9 is a diagram illustrating an emotion map 400 on which a plurality of emotions are mapped. In the emotion map 400, emotions are arranged in concentric circles radiating from the center. The closer to the center of the concentric circles, the more primitive the state of the emotion is arranged. On the outer side of the concentric circles, emotions representing states and actions arising from a state of mind are arranged. Emotion is a concept that also includes affect and mental states. On the left side of the concentric circles, emotions generated from reactions that generally occur in the brain are arranged. On the right side of the concentric circles, emotions that are generally induced by situational judgment are arranged. In the upward and downward directions of the concentric circles, emotions that are generated from reactions that generally occur in the brain and are induced by situational judgment are arranged. Also, on the upper side of the concentric circles, “pleasant” emotions are arranged, and on the lower side, “unpleasant” emotions are arranged. In this way, in the emotion map 400, a plurality of emotions are mapped based on the structure in which emotions are generated, and emotions that are likely to occur at the same time are mapped close to each other.

These emotions are distributed in the 3 o'clock direction of the emotion map 400, and usually go back and forth between relief and anxiety. In the right half of the emotion map 400, situational awareness is superior to internal sensations, resulting in a calm impression.

Since the inside of the emotion map 400 represents the inside of the mind and the outside of the emotion map 400 represents actions, the further one goes to the outside of the emotion map 400, the more visible (manifested in action) the emotion becomes.

Here, human emotions are based on various balances such as posture and blood sugar levels, and show a state of unpleasantness when those balances move away from the ideal, and a state of pleasantness when they approach the ideal. In robots, automobiles, motorcycles, and the like as well, emotions can be created based on various balances such as posture and remaining battery level, so as to show a state of unpleasantness when those balances move away from the ideal, and a state of pleasantness when they approach the ideal. The emotion map may be generated based on, for example, Dr. Mitsuyoshi's emotion map (Research on a speech emotion recognition and brain physiological signal analysis system of affect, Tokushima University, doctoral dissertation: https://ci.nii.ac.jp/naid/500000375379). In the left half of the emotion map, emotions belonging to a region called “reaction” where sensation is dominant are arranged. Also, in the right half of the emotion map, emotions belonging to a region called “situation” where situational awareness is dominant are arranged.

In the emotion map, two emotions that promote learning are defined. One is an emotion around the middle of negative “remorse” and “reflection” on the situation side. That is, it is when a negative emotion such as “I never want to feel this way again” or “I don't want to be scolded anymore” arises in the robot. The other is an emotion around positive “desire” on the reaction side. That is, it is when there is a positive feeling such as “I want more” or “I want to know more”.

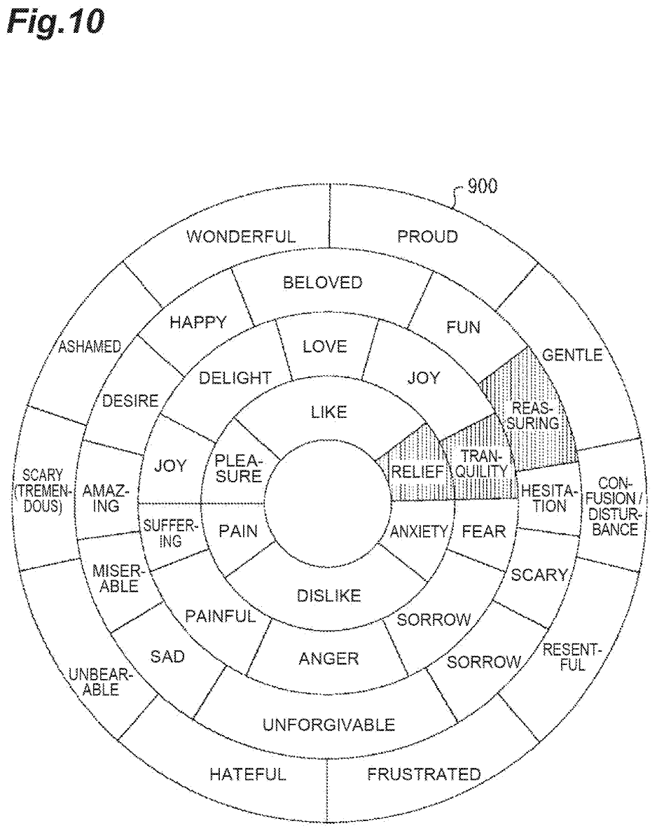

The emotion identification model 59 inputs a user input into a pre-trained neural network, acquires an emotion value indicating each emotion shown in the emotion map 400, and determines the user's emotion. This neural network is pre-trained based on a plurality of learning data that are combinations of user inputs and emotion values indicating each emotion shown in the emotion map 400. Also, this neural network is trained such that emotions arranged close to each other have close values, as in the emotion map 900 shown in FIG. 10. FIG. 10 shows an example in which a plurality of emotions, “relief,” “peace of mind,” and “reassured,” have close emotion values.

Although the system according to the present disclosure has been described above mainly with respect to the functions of the data processing apparatus 12, the system according to the present disclosure is not necessarily implemented in a server. The system according to the present disclosure may be implemented as a general information processing system. The present disclosure may be implemented as, for example, a software program that runs on a personal computer, or an application that runs on a smartphone or the like. The method according to the present disclosure may be provided to a user in a SaaS (Software as a Service) format.

An example form in which the specific processing is performed by one computer 22 has been described, but the technology of the present disclosure is not limited to this, and distributed processing for the specific processing may be performed by a plurality of computers including the computer 22. For example, the data generation model 58 may be provided in an external device of the data processing apparatus 12, and the external device may generate data according to the input data.

An example form in which the specific processing program 56 is stored in the storage 32 has been described, but the technology of the present disclosure is not limited to this. For example, the specific processing program 56 may be stored in a portable computer-readable non-transitory storage medium such as a USB (Universal Serial Bus) memory. The specific processing program 56 stored in the non-transitory storage medium is installed in the computer 22 of the data processing apparatus 12. The processor 28 executes the specific processing according to the specific processing program 56.

Also, the specific processing program 56 may be stored in a storage device such as a server connected to the data processing apparatus 12 via the network 54, and the specific processing program 56 may be downloaded in response to a request from the data processing apparatus 12 and installed in the computer 22.

Note that it is not necessary to store all of the specific processing program 56 in a storage device such as a server connected to the data processing apparatus 12 via the network 54, or to store all of the specific processing program 56 in the storage 32, and a part of the specific processing program 56 may be stored.

As hardware resources for executing the specific processing, various processors shown below can be used. Examples of the processor include a CPU, which is a general-purpose processor that functions as a hardware resource for executing the specific processing by executing software, that is, a program. Also, examples of the processor include a dedicated electric circuit, which is a processor having a circuit configuration specifically designed to execute specific processing, such as an FPGA (Field-Programmable Gate Array), a PLD (Programmable Logic Device), or an ASIC (Application Specific Integrated Circuit). A memory is built in or connected to any of the processors, and any of the processors executes the specific processing by using the memory.

The hardware resource that executes the specific processing may be configured by one of these various processors, or may be configured by a combination of two or more processors of the same type or different types (for example, a combination of a plurality of FPGAs, or a combination of a CPU and an FPGA). Also, the hardware resource that executes the specific processing may be one processor.

As an example of a configuration with one processor, first, there is a form in which one processor is configured by a combination of one or more CPUs and software, and this processor functions as a hardware resource for executing the specific processing. Second, there is a form in which a processor that realizes the functions of an entire system including a plurality of hardware resources for executing the specific processing with one IC chip, as represented by an SoC (System-on-a-chip) or the like, is used. In this way, the specific processing is realized using one or more of the various processors described above as hardware resources.

Furthermore, as a hardware structure of these various processors, an electric circuit in which circuit elements such as semiconductor elements are combined can be used. Also, the specific processing described above is merely an example. Therefore, it goes without saying that unnecessary steps may be deleted, new steps may be added, or the processing order may be changed within a scope that does not depart from the gist.

The description and illustrations shown above are detailed descriptions of the parts related to the technology of the present disclosure, and are merely an example of the technology of the present disclosure. For example, the description regarding the above-described configuration, function, operation, and effect is a description regarding an example of the configuration, function, operation, and effect of the part related to the technology of the present disclosure. Therefore, it goes without saying that unnecessary parts may be deleted, new elements may be added, or replacements may be made to the description and illustrations shown above within a scope that does not depart from the gist of the technology of the present disclosure. Also, in order to avoid confusion and facilitate understanding of the parts related to the technology of the present disclosure, in the description and illustrations shown above, descriptions regarding common general technical knowledge and the like that do not require particular explanation for enabling the implementation of the technology of the present disclosure are omitted.

All documents, patent applications, and technical standards described in this specification are incorporated herein by reference to the same extent as if each individual document, patent application, and technical standard were specifically and individually indicated to be incorporated by reference.

It is to be understood that not all aspects, advantages and features described herein may necessarily be achieved by, or included in, any one particular example. Indeed, having described and illustrated various examples herein, it should be apparent that other examples may be modified in arrangement and detail.

A system including an information input unit, a data analysis unit, a first aid proposal unit, an urgency evaluation unit, and an expert contact unit. The information input unit provides a means for a care staff or a user to input information on a health condition or an injury via a terminal, and the data analysis unit analyzes the received information using natural language processing technology and image recognition technology to determine a type or severity of the injury. The first aid proposal unit provides a means for proposing appropriate first aid based on an analysis result, and the urgency evaluation unit provides a means for evaluating an urgency of an injury using AI and, when a risk is high, sending a notification to a medical expert via the expert contact unit. The expert contact unit provides a means for notifying the medical expert of a user's situation and providing detailed instructions.

In some examples, the information input unit includes an interface for inputting information on a user's health condition or injury via an application installed on a terminal such as a smartphone or a tablet, and the data analysis unit can analyze a text message using natural language processing technology and analyze a photograph using image recognition technology to extract important keywords and analyze a shape of a wound or a degree of bleeding from the photograph.

In some examples, the first aid proposal unit proposes specific first aid such as a method of cleaning with an antiseptic solution or a method of wrapping a bandage based on past medical data and expert knowledge, and the urgency evaluation unit can evaluate an amount of bleeding or a depth of a wound using AI and, when it is determined that an urgency is high, immediately send a notification to a medical expert via the expert contact unit.

An example system for proposing first aid may include circuitry. The circuitry may be configured to: receive data of a text message and an image regarding an injury from a user; determine a state of the injury including a type and severity of the injury by analyzing the received text message using natural language processing technology and analyzing the received image using image recognition technology; propose appropriate first aid to the user based on the state of the injury; infer an urgency of the injury based on the state of the injury; and notify an operator of the state of the injury when it is determined that the urgency is high.

In some examples, receiving the data may include receiving the data via a messenger application.

In some examples, proposing the first aid may include proposing treatment according to a degree of the injury based on past data and medical information.

An example method of proposing first aid may include: receiving data of a text message and an image regarding an injury from a user; determining a state of the injury including a type and severity of the injury by analyzing the received text message using natural language processing technology and analyzing the received image using image recognition technology; proposing appropriate first aid to the user based on the state of the injury; inferring an urgency of the injury based on the state of the injury; and notifying an operator of the state of the injury when it is determined that the urgency is high.

What is claimed is:

1. A system for proposing first aid, the system comprising circuitry,
wherein the circuitry is configured to:
receive data of a text message and an image regarding an injury from a user;
determine a state of the injury including a type and severity of the injury by analyzing the received text message using natural language processing technology and analyzing the received image using image recognition technology;
propose appropriate first aid to the user based on the state of the injury;
infer an urgency of the injury based on the state of the injury; and
notify an operator of the state of the injury when it is determined that the urgency is high.

2. The system according to claim 1, wherein receiving the data includes receiving the data via a messenger application.

3. The system according to claim 1, wherein proposing the first aid includes proposing treatment according to a degree of the injury based on past data and medical information.

4. A method of proposing first aid, the method comprising:
receiving data of a text message and an image regarding an injury from a user;
determining a state of the injury including a type and severity of the injury by analyzing the received text message using natural language processing technology and analyzing the received image using image recognition technology;
proposing appropriate first aid to the user based on the state of the injury;
inferring an urgency of the injury based on the state of the injury; and
notifying an operator of the state of the injury when it is determined that the urgency is high.
